(19) United States
(12) Patent Application Publication
(10) Pub. No.: US 2005/0234654 A1
(43) Pub. Date: Oct. 20, 2005

(54) Detection of evolutionary bottlenecking by dna sequencing as a method to discover genes of value

(76) Inventor: Walter Messier, Longmont, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/522,393

(22) PCT Filed: Aug. 8, 2003
(86) PCT No.: PCT/US03/25027

§ 371 Date: Feb. 17, 2005

(30) Foreign Application Priority Data
Aug. 8, 2002 (US) 60402340

(51) Int. Cl. C12Q 1/68; G06F 19/00; G01N 33/48; G01N 33/50

(52) U.S. Cl. 702/20; 435/6

(57) ABSTRACT

This invention relates to using molecular and evolutionary techniques to identify polynucleotide and polypeptide sequences corresponding to commercially or aesthetically relevant traits in domesticated plants and animals, specifically, a method to detect evolutionary bottleneck sequences.

### TECHNICAL FIELD

This invention relates to using molecular and evolutionary techniques to identify polynucleotide and polypeptide sequences corresponding to commercially or aesthetically relevant traits in domesticated plants and animals.

### BACKGROUND ART

Humans have bred plants and animals for thousands of years, selecting for certain commercially valuable and/or aesthetic traits. Domesticated plants differ from their wild ancestors in such traits as yield, short day length flowering, protein and/or oil content, ease of harvest taste, disease resistance and drought resistance. Domesticated animals differ from their wild ancestors in such traits as fat and/or protein content, milk production, docility, fecundity and time to maturity. At the present time, most genes underlying the above differences are not known, nor, as importantly, are the specific changes that have evolved in these genes to provide these capabilities. Understanding the basis of these differences between domesticated plants and animals and their wild ancestors will provide useful information for maintaining and enhancing those traits. In the case of crop plants, identification of the specific genes that control desired traits will allow direct and rapid improvement in a manner not previously possible.

Although comparison of homologous genes or proteins between domesticated species and their wild ancestors may provide useful information with respect to conserved molecular sequences and functional features, this approach is of limited use in identifying genes responsible for trait differences between domesticated species and their respective ancestral species, as, in many cases, these gene sequences have changed due to selective pressures of domestication.

Prior to Darwin's' publication of On the Origin of Species, biology was a mass of facts, most apparently unrelated and difficult to synthesize into a predictive structure. A fair analogy is that nineteenth century pre-Darwin biology represented a massive postage stamp collection. Darwin's explanation of evolution (and the mechanisms that underlie evolution) proved a much-needed predictive structure. Similarly, today, many specialists in genomics have begun to realize that interpretation of accumulated genomic data is facilitated by use of the predictive power of an evolutionary prism. In U.S. Pat. No. 6,274,319, a method is described that employs algorithms that detect positively selected genes by comparing homologous in protein coding regions between closely related species, as a screening tool to identify and characterize commercially valuable genes. As it is clear that changes in gene regulation can be important for phenotypic variations, and as evidence in the literature of maize domestication (see especially Doebley's work: Doebley, Symp Soc Exp Biol. 1998; 51:127-32; Lukens & Doebley, Mol Biol Evol. 2001 18 (4):627-38; Hubbard, et al. Genetics. 2002 162 (4):1927-35) suggests an important role for regulatory changes during cereal domestication, it is important to also screen non-coding regions for genes whose protein-coding region may not have been measurably affected by domestication, but whose regulation has been, and thus is potentially a commercially valuable gene. Here we provide a different evolutionary analytical approach to screen both protein-coding and non-coding genetic sequences for commercially valuable genes. This novel approach uses algorithms that detect “evolutionary bottlenecking” as a screening tool to identify and characterize commercially valuable genes.

An evolutionary bottleneck is a severe decline in the size of a population, leaving a very few individuals for some period, followed by an increase in the surviving population. Evolutionary bottlenecks can result from random forces of nature, such as disease or climate change, or directed forces, such as domestication of crops by humans. Evolutionary bottlenecks result in decreased allelic variability.

Several papers detail methods to detect evolutionary bottlenecks by looking for reduced allelic variability in a particular gene in a population or species. Others have used some version of bottleneck analysis to look for evidence of narrowing of gene pools in wild plant species (see for example Kwon, J. A. & Morden, C. W. 2002 Molecular Ecology 11 (6): 991), or in domesticated plants vs. their wild ancestors [See, for example, Van Cutsem et al. 2003 Theor. Appl. Genet. (advance online publication) or Eyre-Walker, A. et al. 1998 PNAS 95: 441-446]. These attempts were purely academic exercises in understanding some aspect of population genetics. None of these earlier papers used evolutionary bottleneck detection as a screening tool to systematically identify commercially valuable genes, such as genes responsible for the traits enhanced or imposed by domestication.

The identification of genes whose allelic variation has been constricted by the evolutionary bottleneck such as that imposed by domestication of plants or animals by humans, to fix unique, enhanced, or altered functions compared to homologous ancestral genes could be used to further enhance these functions, through development of genetically modified organisms or of agents to modulate these functions.

### DISCLOSURE OF INVENTION

The present invention provides a method for identifying polynucleotide and polypeptide sequences that have undergone an evolutionary bottleneck, which are associated with commercial or aesthetic traits. The invention uses comparative genomics to identify specific genes which may be associated with, and thus responsible for, structural, biochemical or physiological conditions, such as commercially or aesthetically relevant traits, and using the information obtained from these genes to develop organisms with enhanced traits of interest or agents to enhance or in other ways modulate such traits. In one preferred embodiment, a polynucleotide or polypeptide of a domesticated plant or animal has, because of human artificial selection, undergone an evolutionary bottleneck when compared in its respective ancestor. One example of this embodiment is that the polynucleotide or polypeptide may be associated with enhanced crop yield as compared to the ancestor. Other examples include short day length flowering (i.e., flowering only if the daily period of light is shorter than some critical length), protein content, oil content, ease of harvest, taste, drought resistance and disease resistance. The present invention can thus be useful in gaining insight into the genes and molecular mechanisms that underlie functions or traits in domesticated organisms. This information can be useful in utilizing the polynucleotide or a modification of the polynucleotide, or agents identified in assays incorporating the polynucleotide or its encoded polypeptide, so as to further enhance the function or trait. For example, a polynucleotide determined to be responsible for improved crop yield could be subjected to random or directed mutagenesis, followed by testing of the mutant genes to identify those that further enhance the trait. As another example, a copy or a modified copy of such a yield-affecting polynucleotide may be transformed into a crop plant to enhance a relevant trait.

Accordingly, in one aspect, the invention provides method for identifying a polynucleotide sequence, wherein the polynucleotide sequence may be associated with a commercially or aesthetically relevant trait, comprising:
a) aligning homologous nucleotide sequences of at least two individual organisms, wherein said at least two individual organisms are selected from the group consisting of individual organisms of a single strain, individual organisms of different strains, individual organisms of the same species, individual organisms of different species, and any combination of the foregoing, wherein one nucleotide sequence is associated with an individual organism exhibiting said commercially or aesthetically relevant trait; and
b) detecting a region of polynucleotide sequence for which the number of nucleotide differences/site indicates an evolutionary bottleneck;
   whereby a polynucleotide sequence associated with a commercially or aesthetically relevant trait of said organism may be identified

In another aspect, the invention provides method for identifying a polynucleotide sequence of a domesticated organism, wherein the polynucleotide sequence may be associated with a commercially or aesthetically relevant trait that is unique, enhanced or altered in the domesticated organism as compared to other domesticated or ancestral species of the domesticated organism, comprising:
a) aligning homologous protein-coding nucleotide sequences of at least two individual organisms, wherein said at least two individual organisms are selected from the group consisting of individual organisms of a single strain, individual organisms of different strains, individual organisms of the same species, individual organisms of different species, and any combination of the foregoing, wherein one nucleotide sequence is associated with an domesticated organism exhibiting said commercially or aesthetically relevant trait; and
b) detecting a region of polynucleotide sequence for which the number of nucleotide differences/site indicates an evolutionary bottleneck;
   whereby a polynucleotide sequence associated with a commercially or aesthetically relevant trait that is unique, enhanced or altered in the domesticated organism as compared to other domesticated or ancestral species of said organism may be identified.

In a further aspect, the invention provides a method for identifying a polynucleotide sequence encoding a polypeptide, wherein the polypeptide may be associated with a commercially or aesthetically relevant trait comprising:
a) aligning homologous protein-coding nucleotide sequences of at least two individual organisms, wherein said at least two individual organisms are selected from the group consisting of individual organisms of a single strain, individual organisms of different strains, individual organisms of the same species, individual organisms of different species, and any combination of the foregoing, wherein one nucleotide sequence encodes a polypeptide associated with an domesticated organism exhibiting said commercially or aesthetically relevant trait; and
b) detecting a region of polynucleotide sequence for which the number of nucleotide differences/site indicates an evolutionary bottleneck;
   whereby a polynucleotide sequence associated with a commercially or aesthetically relevant trait of said organism may be identified.

In yet a further aspect, the invention provides a method for identifying a polynucleotide sequence encoding a polypeptide of a domesticated organism, wherein the polynucleotide sequence may be associated with a commercially or aesthetically relevant trait that is unique, enhanced or altered in the domesticated organism as compared to other domesticated or ancestral species of the domesticated organism, comprising
a) aligning homologous protein-coding nucleotide sequences of at least two individual organisms, wherein said at least two individual organisms are selected from the group consisting of individual organisms of a single strain, individual organisms of different strains, individual organisms of the same species, individual organisms of different species, and any combination of the foregoing, wherein one nucleotide sequence encodes a polypeptide associated with an domesticated organism exhibiting said commercially or aesthetically relevant trait; and
b) detecting a region of polynucleotide sequence for which the number of nucleotide differences/site indicates an evolutionary bottleneck;
   whereby a polynucleotide sequence associated with a commercially or aesthetically relevant trait unique, enhanced or altered in the domesticated organism as compared to other domesticated or ancestral species of said organism may be identified.

In a still further aspect, the invention provides a method for identifying a regulatory element comprising:

comprising:

a) aligning homologous nucleotide sequences of at least about two strains and/or individuals of a single strain of said organism; and
b) a) aligning homologous nucleotide sequences of at least two individual organisms, wherein said at least two individual organisms are selected from the group consisting of individual organisms of a single strain, individual organisms of different strains, individual organisms of the same species, individual organisms of different species, and any combination of the foregoing, wherein one nucleotide sequence encodes a polypeptide associated with an domesticated organism exhibiting said commercially or aesthetically relevant trait; and;
c) determining that the region identified in b) is a non-coding region, whereby a regulatory element is identified.

In some aspects, the identifying the number of nucleotide differences/site referred to is calculated by

$$π = 1 / [ n ( n - 1 ) / 2 ] {\sum }_{i < j} Πⅈj / L ,$$

where i and j represent any two sequences being compared in a series of sequences and L=sequence length.

In other aspects, the methods further comprise determining if the region displays a signature of positive selection, which in some aspects comprises calculating a Ka/Ks value.

In some aspects the method is performed in an automated pipeline.

In the further aspects, the at least two strains and/or individuals of a single strain is at least ten strains and/or individuals of a single strain.

In the further aspects, the at least two strains and/or individuals of a single strain is at least fifteen strains and/or individuals of a single strain.

In still further aspects, the invention provides a method for identifying an agent which may modulate a commercially or aesthetically relevant trait that is unique, enhanced or altered in the domesticated organism as compared to other domesticated or ancestral species of the domesticated organism, said method comprising contacting at least one candidate agent with a cell, model system or transgenic plant or animal that expresses a polynucleotide sequence that is an evolutionary bottleneck, wherein the agent is identified by its ability to modulate function of the polypeptide encoded by the polynucleotide.

In still further aspects, the invention provides a method for correlating a nucleotide sequence which is an evolutionary bottleneck to a commercially or aesthetically relevant trait that is unique, enhanced or altered in a domesticated organism, comprising:
a) identifying a nucleotide sequence which is an evolutionary bottleneck; and
b) analyzing the functional effect of the presence or absence of the identified sequence in the domesticated organism or in a model system.

Also provided is a method for automated comparison of a large amount of nucleotide sequence of two or more strains of an organism, said method comprising: a) aligning homologous nucleotide sequences of two or more strains and/or individuals of a single strain of the crop or said organism, and b) detecting regions of polynucleotide sequence for which the number of nucleotide differences/site indicates an evolutionary bottleneck.

In another aspect, the subject invention provides a method to make improved plants or animals by transforming cells or said plant or animal or otherwise inserting a copy or modified copy of a polynucleotide sequence identified using the methods herein.

In another aspect, the subject invention provides a method for correlating a nucleotide sequence which has undergone an evolutionary bottleneck to a commercially or aesthetically relevant trait that is unique, enhanced or altered in a domesticated organism, comprising: a) identifying a nucleotide sequence which has undergone an evolutionary bottleneck according to the methods described herein; and b) analyzing the functional effect of the presence or absence of the identified sequence in the domesticated organism or in a model system.

The domesticated plants used in the subject methods can be but are not limited to maize, wheat, barley, rye, millet, chickpea, lentil, flax, olive, fig almond, pistachio, walnut, beet, parsnip, citrus fruits, including, but not limited to, orange, lemon, lime, grapefruit, tangerine, minneola, and tangelo; sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, and woody plants such as coniferous and deciduous trees. The relevant trait can be any commercially or aesthetically relevant trait such as yield, short day length flowering, protein content, oil content, drought resistance, taste, ease of harvest or disease resistance.

The domesticated animals used in the subject methods can be any domesticated animal. The relevant trait could, for example, be fat content, protein content, milk production, time to maturity, fecundity, docility or disease resistance and disease susceptibility.

### DETAILED DESCRIPTION OF THE INVENTION

The present invention utilizes comparative genomics to identify specific polynucleotides and polypeptides associated with, and thus may contribute to or be responsible for, commercially or aesthetically relevant traits in.

In a preferred embodiment, the methods described herein can be applied to identify the genes that control traits of interest in agriculturally important domesticated plants. Humans have bred domesticated plants for several thousand years without knowledge of the genes that control these traits. Knowledge of the specific genetic mechanisms involved would allow much more rapid and direct intervention at the molecular level to create plants with desirable or enhanced traits or to screen for agents with which plants could be treated to enhance specific traits.

Humans, through artificial selection, have imposed evolutionary bottlenecks on crop plants. These evolutionary bottlenecks are reflected in reduced nucleotide diversity in the genes critical to traits important in domestication and this reduced nucleotide diversity can be used as a signal to identify these important genes. It has been found that only a few genes, e.g., 10-15 per species, control traits of commercial interest in domesticated crop plants. These few genes have been exceedingly difficult to identify through standard methods of plant molecular biology. Yet, a majority of these genes are likely to show evidence of an evolutionary bottleneck, imposed by domestication. Thus, the evolutionary bottlenecking screening method described herein should identify a majority of the genes controlling traits of interest.

For any crop plant of interest, genomic DNA can be isolated from at least two, and preferably multiple, strains of the crop and/or at least two, and preferably multiple, individuals of a single strain of the crop. The isolated DNA can then be sequenced by any of the methods known to those skilled in the art. Alternatively, the skilled artisan can access commercially and/or publicly available genomic databases rather than isolating and sequencing DNA. Homologous DNA sequences from each of the strains and/or individuals can then be aligned by any of the methods well known to those skilled in the art.

Once homologous sequences are aligned, the number of nucleotide differences/site (π) can be estimated. One formula for determining n for a number of sequences (n) is

$$π = 1 / [ n ( n - 1 ) / 2 ] {\sum }_{i < j} Πⅈj / L$$

Where i and j represent any two sequences being compared in a series of sequences and L=sequence length.

Any suitable index of nucleotide diversity could be used, although for the purposes of the invention, π is the preferred index. However, this invention is not limited only to use of π. Examples of other possible indices include P, the fraction of nucleotides shared between homologous sequences, and θ, the silent site nucleotide diversity.
P=n_{xy}/√nₓn_{y}
where n_{xy} are the number of nucleotides shared (excluding insertions and deletions) by sequences x and y, and n_{y} and n_{y} are the number of nucleotides of sequences x and y, respectively.
θ=s(aₙ₋₁)⁻¹m⁻¹
where n=number of sequences in the sample, s is the number of polymorphic silent sites in the sample, m is the number of sites in the sample, and a is given by

$${\sum }_{i = 1}^{n - 1} 1 / i .$$

Genes with low nucleotide diversity are chosen for further analysis. π can theoretically range from 0.0000 (0.0%), which would indicate no nucleotide diversity (i.e., identical sequences or sequence identity) to 1.000 (100%) which would signify two totally different (and thus, non-homologous) sequences. π values are available for several specific genes, but, no conclusive data are available for most species regarding the expected range of species-specific it values. However, as the skilled practitioner determines π values for more and more sequences of a species, the full range of π values as well as the unusually low π values of interest will be refined. For any species, π values can be determined empirically by those skilled in the art, and it values that are unusually low will be readily ascertained one skilled in the art.

One preferred embodiment for the estimation of π is to use an automated informatics pipeline, in which homologous sequences are aligned, and π is calculated for sections of the aligned homologous sequences. The optimal length of these sections of sequence to be used in estimating π must be determined empirically, but a reasonable starting length might be about 1000 bp. In practice, the optimal length may be shorter or longer; however, the optimal length must be determined for each comparison. In the case of an automated procedure for large scale nucleotide comparison, a reasonable starting length might be for example, about 10,000 bp. The starting length is not meant to limit the use actual optimal length, once an optimal length has been determined. This approach requires no prior knowledge about the sequence being examined, i.e., the positions and lengths of coding sequence and regulatory regions. This adds power to the invention, in that we can identify regions of sequence that were bottlenecked during domestication without any assumptions about the type of gene, its function, or its position on the chromosome or within a QTL. We thus can ‘cast a wide net’.

As π values are estimated sequentially (or successively) along the DNA sequence, an overlapping strategy is useful, in which, after estimating π values along the sequences, the frame of reference is shifted by a predetermined number of base pairs, say 50 bp. As little data on expected values of π currently exists, the optimal number of base pairs to shift to a new frame of reference must be determined empirically for each species examined. Similarly, the optimal length of sequence to estimate π values will also be determined for each species examined.

As a database of π values is amassed for each species examined, the most crucial low values of π for that particular species will become clear. This is fundamentally an iterative process; thus the most critical π values will be refined as data accumulates.

Nucleotide sequences with low π values may then evaluated using standard molecular and transgenic plant methods to determine if they play a role in the traits of commercial or aesthetic interest. The genes of interest are then manipulated by, e.g., random or site-directed mutagenesis, to develop new, improved varieties, subspecies, strains or cultivars. Alternatively, the polynucleotide of interest is used to develop screening assays to identify agents with the ability to modulate the polynucleotides or the polypeptides encoded by such polynucleotides to achieve a desired effect.

Similarly, the methods described herein can be applied to domesticated animals including pigs, cattle, horses, dogs, cats and any other domesticated animals. Cattle and horses, especially, represent important commercial interests. As with plants, humans have bred animals for thousands of years, and those intense selection pressures will be reflected in evolutionary bottlenecks. Again, genomic DNA can be isolated from at least two, or preferably multiple strains and/or individuals of a single strain of the animal. The isolated DNA can then be sequenced by any of the methods known to those skilled in the art. Homologous DNA sequences from each of the strains and/or individuals can then be aligned by any of the methods well known to those skilled in the art. Alternatively, the skilled artisan can access commercially and/or publicly available genomic databases rather than isolating and sequencing DNA.

For homologous sequences, the number of nucleotide differences/site (π) can be calculated, and those genes with low π estimates selected. These genes are then evaluated using standard molecular and transgenic animals methods to determine if they play a role in the traits of commercial or aesthetic interest. Those genes can then be manipulated to develop new, improved animal varieties or subspecies, or agents to enhance or modulate the trait of interest.

The practice of the present invention employs, unless otherwise indicated, conventional techniques of molecular biology, genetics and molecular evolution, which are within the skill of the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Current Protocols in Molecular Biology” (F. M. Ausubel et al., eds., 1987); “PCR: The Polymerase Chain Reaction”, (Mullis et al., eds., 1994); “Molecular Evolution”, (Li, 1997).

Definitions

As used herein, a “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides. The terms “polynucleotide” and “nucleotide sequence” are used interchangeably.

As used herein, a “gene” refers to a polynucleotide or portion of a polynucleotide comprising a sequence that encodes a protein. It is well understood in the art that a gene also comprises non-coding sequences, such as 5′ and 3′ flanking sequences (such as promoters, enhancers, repressors, and other regulatory sequences) as well as introns.

The terms “polypeptide,” “peptide,” and “protein” are used interchangeably herein to refer to polymers of amino acids of any length. These terms also include proteins that are post-translationally modified through reactions that include glycosylation, acetylation and phosphorylation.

The term “domesticated organism” refers to an individual living organism or population of same, a species, subspecies, variety, cultivar or strain, that has been subjected to artificial selection pressure and developed a commercially or aesthetically relevant trait. In some preferred embodiments, the domesticated organism is a plant selected from the group consisting of corn, wheat, rice, sorghum, tomato or potato, or any other domesticated plant of commercial interest. In other preferred embodiments, the domesticated organism is an animal selected from the group consisting of cattle, horses, pigs, cats and dogs.

The term “wild ancestor” or “ancestor” means a forerunner or predecessor organism, species, subspecies, variety, cultivar or strain from which a domesticated organism, species, subspecies, variety, cultivar or strain has evolved. A domesticated organism can have one or more than one ancestor. Typically, domesticated plants can have one or a plurality of ancestors, while domesticated animals usually have only a single ancestor.

The term “commercially or aesthetically relevant trait” is used herein to refer to traits that exist in domesticated organisms such as plants or animals whose analysis could provide information (e.g., physical or biochemical data) relevant to the development of agents that can modulate the polypeptide responsible for the trait. The commercially or aesthetically relevant trait can be unique, enhanced or altered relative to the ancestor. By “altered,” it is meant that the relevant trait differs qualitatively or quantitatively from traits observed in the ancestor.

The term “evolutionary bottleneck” evolutionary bottleneck refers to an event that causes a severe decline in the size of a population, leaving a very few individuals for some period, followed by an increase in the surviving population. Evolutionary bottlenecks result in decreased allelic variability. Bottlenecking events can result from random forces of nature, such as disease or climate change, or directed forces, such as domestication of crops by humans. One formula for determining π for a number of sequences (n) is

$$π = 1 / [ n ( n - 1 ) / 2 ] {\sum }_{i < j} Πⅈj / L$$

Where i and j represent any two sequences being compared in a series of sequences and L=sequence length.

The term “resistant” means that an organism exhibits an ability to avoid, or diminish the extent of, a disease condition and/or development of the disease, preferably when compared to non-resistant organisms.

The term “susceptibility” means that an organism fails to avoid, or diminish the extent of, a disease condition and/or development of the disease condition, preferably when compared to an organism that is known to be resistant.

It is understood that resistance and susceptibility vary from individual to individual, and that, for purposes of this invention, these terms also apply to a group of individuals within a species, and comparisons of resistance and susceptibility generally refer to overall, average differences between species, although intra-specific comparisons may be used.

The term “homologous” or “homologue” or “ortholog” is known and well understood in the art and refers to related sequences that share a common ancestor and is determined based on degree of sequence identity. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to, (a) degree of sequence identity; (b) same or similar biological function. Preferably, both (a) and (b) are indicated. The degree of sequence identity may vary, but is preferably at least 50% (when using standard sequence alignment programs known in the art), more preferably at least 60%, more preferably at least about 75%, more preferably at least about 85%. Homology can be determined using software programs readily available in the art, such as those discussed in Current Protocols in Molecular Biology (F. M. Ausubel et al., eds., 1987) Supplement 30, section 7.718, Table 7.71.

The term “nucleotide change” refers to nucleotide substitution, deletion, and/or insertion, as is well understood in the art.

“Housekeeping genes” is a term well understood in the art and means those genes associated with general cell function, including but not limited to growth, division, stasis, metabolism, and/or death. “Housekeeping” genes generally perform functions found in more than one cell type. In contrast, cell-specific genes generally perform functions in a particular cell type and/or class.

The term “agent”, as used herein, means a biological or chemical compound such as a simple or complex organic or inorganic molecule, a peptide, a protein or an oligonucleotide that modulates the function of a polynucleotide or polypeptide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

The term “to modulate function” of a polynucleotide or a polypeptide means that the function of the polynucleotide or polypeptide is altered in the presence of an agent compared to the absence of the agent. Modulation may occur on any level that affects function. A polynucleotide or polypeptide function may be direct or indirect, and measured directly or indirectly.

A “function of a polynucleotide” includes, but is not limited to, replication; translation; expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function.

A “function of a polypeptide” includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

The term “target site” means a location in a polypeptide which can be a single amino acid and/or is a part of, a structural and/or functional motif, e.g., a binding site, a dimerization domain, or a catalytic active site. Target sites may be useful for direct or indirect interaction with an agent, such as a therapeutic agent.

The term “molecular difference” includes any structural and/or functional difference. Methods to detect such differences, as well as examples of such differences, are described herein.

A “functional effect” is a term well known in the art, and means any effect which is exhibited on any level of activity, whether direct or indirect.

The term “ease of harvest” refers to plant characteristics or features that facilitate manual or automated collection of structures or portions (e.g., fruit, leaves, roots) for consumption or other commercial processing.

The term “quantitative trait locus” or (plural) “quantitative trait loci” refers to a chromosomal region (or regions if plural) shown through gene mapping techniques to contain a gene or genes associated with a complex or polygenic (encoded by more than one gene) trait.

The terms “evolutionarily significant change” and “adaptive evolutionary change” refer to one or more nucleotide or peptide sequence change(s) between two organisms, species, subspecies, varieties, cultivars and/or strains that may be attributed to either relaxation of selective pressure or positive selective pressure. One method for determining the presence of an evolutionarily significant change is to apply a K_{A}/K_{S}-type analytical method, such as to measure a K_{A}/K_{S} ratio. Typically, a K_{A}/K_{S} ratio of greater than 1.0 is considered to be an evolutionarily significant change.

Strictly speaking, K_{A}/K_{S} ratios of exactly 1.0 are indicative of relaxation of selective pressure (neutral evolution), and K_{A}/K_{S} ratios greater than 1.0 are indicative of positive selection. However, it is commonly accepted that the ESTs in GenBank and other public databases often suffer from some degree of sequencing error, and even a few incorrect nucleotides can influence K_{A}/K_{S} ratios. For this reason, polynucleotides with K_{A}/K_{S} ratios as low as 0.75 can be selected and carefully resequenced and re-evaluated for either relaxation of selective pressure of positive selective pressure.

The term “positively selected” means an evolutionarily significant change in a particular organism, species, subspecies, variety, cultivar or strain that results in an adaptive change as compared to other related organisms. An example of a positive evolutionarily significant change is a change that has resulted in enhanced yield in crop plants. As stated above, positive selection is indicated by a K_{A}/K_{S} ratio greater than 1.0. With increasing preference, the KA/KS value is greater than 1.25, 1.5 and 2.0.

For the purposes of this invention, the source of the polynucleotide from the domesticated plant or animal can be any suitable source, e.g., genomic sequences or cDNA sequences. Preferably, genomic sequences are compared. Genomic sequences can be obtained from available private, public and/or commercial databases such as those described herein. These databases serve as repositories of the molecular sequence data generated by ongoing research efforts. Alternatively, DNA sequences may be obtained from, for example, sequencing of genomic DNA isolated from tissues of domesticated plants and/or animals, or after PCR amplification from such genomic DNA, or from commercially available genomic DNA libraries according to methods well known in the art. In one embodiment, genomic DNA is PCR-amplified from a chromosomal region corresponding to a quantitative trait locus (QTL) associated with a trait of interest.

Alternatively, cDNA sequences may be used, although this applies the invention for screening coding sequences only. cDNA libraries used for the sequence comparison of the present invention can be constructed using conventional cDNA library construction techniques that are explained fully in the literature of the art. Total mRNAs are used as templates to reverse-transcribe cDNAs. Transcribed cDNAs are subcloned into appropriate vectors to establish a cDNA library. The established cDNA library can be maximized for full-length cDNA contents, although less than full-length cDNAs may be used. Furthermore, the sequence frequency can be normalized according to, for example, Bonaldo et al. (1996) Genome Research 6:791-806. cDNA clones randomly selected from the constructed cDNA library can be sequenced using standard automated sequencing techniques. Preferably, full-length cDNA clones are used for sequencing. Either the entire or a large portion of cDNA clones from a cDNA library may be sequenced although it is also possible to practice some embodiments of the invention by sequencing as little as two cDNA clones.

In one embodiment of the present invention, cDNA clones to be sequenced can be pre-selected according to their expression specificity. In order to select cDNAs corresponding to active genes that are specifically expressed, the cDNAs can be subject to subtraction hybridization using mRNAs obtained from other organs, tissues or cells of the same animal.

Under certain hybridization conditions with appropriate stringency and concentration, those cDNAs that hybridize with non-tissue specific mRNAs and thus likely represent “housekeeping” genes will be excluded from the cDNA pool. Accordingly, remaining cDNAs to be sequenced are more likely to be associated with tissue-specific functions. For the purpose of subtraction hybridization, non-tissue-specific mRNAs can be obtained from one organ, or preferably from a combination of different organs and cells. The amount of non-tissue-specific mRNAs is maximized to saturate the tissue-specific cDNAs.

Alternatively, information from online databases can be used to select or give priority to cDNAs that are more likely to be associated with specific functions. For example, the ancestral cDNA candidates for sequencing can be selected by PCR using primers designed from candidate domesticated organism cDNA sequences. Candidate domesticated organism cDNA sequences are, for example, those that are only found in a specific tissue, such as skeletal muscle, or that correspond to genes likely to be important in the specific function. Such tissue-specific cDNA sequences may be obtained by searching online sequence databases in which information with respect to the expression profile and/or biological activity for cDNA sequences may be specified.

In some embodiments, the cDNA is prepared from mRNA obtained from a tissue at a determined developmental stage, or a tissue obtained after the organism has been subjected to certain environmental conditions.

DNA sequences may be obtained using methods standard in the art, such as PCR methods (using, for example, GeneAmp PCR System 9700 thermocyclers (Applied Biosystems, Inc.)).

### GENERAL METHODS OF THE INVENTION

The underlying approach to genomics and gene/target identification described herein is based on strategies derived from modern evolutionary biology. Evolutionary signatures (which can now be identified by sophisticated mathematical algorithms) may be searched as a rapid means to gene identification.

The initial steps of crop or animal domestication likely included an evolutionary bottleneck, resulting in more limited genetic variation among crop plants or domesticated animals. In order to detect such bottlenecks in a given organism, a set of nucleotide sequences from at least two, and preferably multiple, strains of the organism or individuals of a single strain of the organism is required. The number of individuals required for a robust test varies (partly as a result of within-species variability), although the inventors believe that in some cases, two or a few sequences may be adequate, in most cases 10 to 15 individuals are preferred. The power of the evolutionary bottlenecking screen is increased by sampling individuals from a broad range of phylogenetic and biogeographic diversity.

We predict that as a result of domestication, allelic diversity at selected chromosomal loci (whether protein-coding or regulatory) will be reduced, because of the severe bottleneck imposed by domestication. Some estimates suggest that domestication of maize, for example, occurred in a period lasting only tens of years, with domesticators narrowing the population to only a few hundred plants (the evolutionary bottleneck event) that were then propagated. The present invention makes use of this prediction.

After obtaining and sequencing the DNA as described above, the evolutionary bottleneck analysis is conducted. Two or more homologous sequences are aligned and the number of nucleotide differences/site (π) is calculated along corresponding subsections of the aligned sequences from one end of the subject DNA through to the other. π is calculated as

$$π = 1 / [ n ( n - 1 ) / 2 ] {\sum }_{i < j} Πⅈj / L .$$

Where i and j represent any two sequences being compared in a series of sequences and L=sequence length.

Genes with low π values are chosen. π can theoretically range from 0.0000 (0.0%), which would indicate no nucleotide diversity (i.e., identical sequences or sequence identity) to 1.000 (100%) which would signify two totally different (and thus, non-homologous) sequences. π values are available for several specific genes, but, no conclusive data are available for most species regarding the expected range of species-specific π values. However, as the skilled practitioner determines π values for more and more sequences of a species, the full range of π values as well as the unusually low it values of interest will be refined. For any species, π values can be determined empirically by those skilled in the art, and π values that are unusually low will be obvious to one skilled in the art.

π values provide a particularly useful index, and can easily be calculated in a high throughput environment (i.e., by automating a suitable algorithm).

Any region (whether coding or non-coding) that displays relatively low π values (for example, both between modern and ancestral rice species, or within modern rice species), is chosen for further analysis. Such regions are extremely likely to be the result of evolutionary bottlenecking during domestication. In some cases, such a bottlenecked region will also display the signature of positive selection (e.g., Ka/Ks>1), or, if the region is non-coding, it may be an important regulatory element. Note that this approach does not rely on prior identification of a region as a regulatory element. Thus, we can expect to identify previously unknown regulatory elements. This approach will of course work for any stretch of DNA, regardless of the function of that stretch, including intergenic “junk DNA”, promoters, enhancers, introns, and so on.

There is a clear distinction between the identification of genes positively selected during domestication, as described in U.S. Pat. No. 6,274,319, (or as Vigouroux et al. 2002 PNAS 99: 9650-9655 have attempted using a different strategy that involved screening for microsatellites), and the method discussed here. The method described here is based upon the detection of evolutionary bottlenecks—independent of whether or not the same region has been positively selected. The detection of bottlenecks represents a very powerful, novel strategy for identifying genes of agricultural and commercial value.

Genes or other polynucleotide sequences identified by the present invention may be utilitzed as probes to identify polynucleotides that hybridize under stringent hybridization conditions with the identified polynucleotide. A polynucleotide identified by the present invention can include an isolated natural gene or a homologue thereof, the latter of which is described in more detail below. A polynucleotide identified by the present invention can include one or more regulatory regions, full-length or partial coding regions, or combinations thereof. The minimal size of a polynucleotide of the present invention is the minimal size that can form a stable hybrid with one of the aforementioned genes under stringent hybridization conditions. Suitable and preferred plants are disclosed above.

In accordance with the present invention, an isolated polynucleotide is a polynucleotide that has been removed from its natural milieu (i.e., that has been subject to human manipulation). As such, “isolated” does not reflect the extent to which the polynucleotide has been purified. An isolated polynucleotide can include DNA, RNA, or derivatives of either DNA or RNA.

An isolated polynucleotide identified by present invention can be obtained from its natural source either as an entire (i.e., complete) gene or a portion thereof capable of forming a stable hybrid with that gene. An isolated polynucleotide can also be produced using recombinant DNA technology (e.g., polymerase chain reaction (PCR) amplification, cloning) or chemical synthesis. Isolated polynucleotides include natural polynucleotides and homologues thereof, including, but not limited to, natural allelic variants and modified polynucleotides in which nucleotides have been inserted, deleted, substituted, and/or inverted in such a manner that such modifications do not substantially interfere with the polynucleotide's ability to form stable hybrids under stringent conditions with natural gene isolates.

A polynucleotide homologue can be produced using a number of methods known to those skilled in the art (see, for example, Sambrook et al., ibid.). For example, polynucleotides can be modified using a variety of techniques including, but not limited to, classic mutagenesis techniques and recombinant DNA techniques, such as site-directed mutagenesis, chemical treatment of a polynucleotide to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of polynucleotides and combinations thereof. Polynucleotide homologues can be selected from a mixture of modified nucleic acids by screening for the function of the polypeptide encoded by the nucleic acid (e.g., ability to elicit an immune response against at least one epitope of the polypeptide encoded by the polynucleotide, ability to promote enhanced economic productivity in a transgenic plant containing the polynucleotide) and/or by hybridization with a gene from a domesticated organism or its wild ancestor.

An isolated polynucleotide identified by present invention can include a nucleic acid sequence that encoding at least one corresponding polypeptide. Although the phrase “polynucleotide” primarily refers to the physical polynucleotide and the phrase “nucleic acid sequence” primarily refers to the sequence of nucleotides on the polynucleotide, the two phrases can be used interchangeably, especially with respect to a polynucleotide, or a nucleic acid sequence, being capable of encoding a polypeptide. As heretofore disclosed, polypeptides of the present invention include, but are not limited to, polypeptides that are full length proteins, polypeptides that are partial proteins, fusion polypeptides, multivalent protective polypeptides and combinations thereof.

At least certain polynucleotides identified by the present invention encode polypeptides that selectively bind to immune serum derived from an animal that has been immunized with a polypeptide from which the polynucleotide was isolated.

A preferred polynucleotide of the present invention, when present in a suitable plant, is capable of increasing the yield of the plant. As will be disclosed in more detail below, such a polynucleotide can be, or encode, an antisense RNA, a molecule capable of triple helix formation, a ribozyme, or other nucleic acid-based compound.

A polynucleotide complement of any nucleic acid sequence identified by the present invention refers to the nucleic acid sequence of the polynucleotide that is complementary to (i.e., can form a complete double helix with) the strand for which the sequence is cited. It is to be noted that a double-stranded nucleic acid molecule identified by present invention for which a nucleic acid sequence has been determined for one strand also comprises a complementary strand. As such, polynucleotides identified by the present invention, which can be either double-stranded or single-stranded, include those polynucleotides that form stable hybrids under stringent hybridization conditions with either a given sequence and/or with the complement of that sequence. Methods to deduce a complementary sequences are known to those skilled in the art. Preferred is a polynucleotide that includes a nucleic acid sequence having at least about 65 percent, preferably at least about 70 percent, more preferably at least about 75 percent, more preferably at least about 80 percent, more preferably at least about 85 percent, more preferably at least about 90 percent and even more preferably at least about 95 percent homology with the corresponding region(s) of the nucleic acid sequence encoding at least a portion of a corresponding polypeptide. Particularly preferred is a polynucleotide capable of encoding at least a portion of a polypeptide that naturally is present in plants.

A preferred polynucleotide identified by the present invention includes at least a portion of nucleic acid sequence that is capable of hybridizing (i.e., that hybridizes under stringent hybridization conditions) to an gene identified by the present invention, as well as a polynucleotide that is an allelic variant of any of those polynucleotides. Such preferred polynucleotides can include, but are not limited to, a full-length gene, a full-length coding region, a polynucleotide encoding a fusion polypeptide, and/or a polynucleotide encoding a multivalent protective compound, including polynucleotides that have been modified to accommodate codon usage properties of the cells in which such polynucleotides are to be expressed.

Knowing the nucleic acid sequences of certain polynucleotides identified by the present invention allows one skilled in the art to, for example, (a) make copies of those polynucleotides, (b) obtain polynucleotides including at least a portion of such polynucleotides (e.g., polynucleotides including full-length genes, full-length coding regions, regulatory control sequences, truncated coding regions), and (c) obtain corresponding polynucleotides for other plants, particularly since, knowledge of polynucleotides identified by the present invention will enable the isolation of polynucleotides in other domesticated organisms and their wild ancestors. Such polynucleotides can be obtained in a variety of ways including screening appropriate expression libraries with antibodies; traditional cloning techniques using oligonucleotide probes of the present invention to screen appropriate libraries or DNA; and PCR amplification of appropriate libraries or DNA using suitable oligonucleotide primers. Preferred libraries to screen or from which to amplify polynucleotides include libraries such as genomic DNA libraries, BAC libraries, YAC libraries, cDNA libraries prepared from isolated plant tissues, including, but not limited to, stems, reproductive structures/tissues, leaves, roots, and tillers; and libraries constructed from pooled cDNAs from any or all of the tissues listed above. In the case of rice, BAC libraries, available from Clemson University, are preferred. Similarly, preferred DNA sources to screen or from which to amplify polynucleotides include plant genomic DNA. Techniques to clone and amplify genes are disclosed, for example, in Sambrook et al., ibid. and in Galun & Breiman, TRANSGENIC PLANTS, Imperial College Press, 1997.

Polynucleotides that are oligonucleotides capable of hybridizing, under stringent hybridization conditions, with complementary regions of other, preferably longer, polynucleotides identified by the present invention can also be identified. Oligonucleotides identified by the present invention can be RNA, DNA, or derivatives of either. The minimal size of such oligonucleotides is the size required to form a stable hybrid between a given oligonucleotide and the complementary sequence on another polynucleotide.

The minimal size of a protein homolog of the present invention is a size sufficient to be encoded by a nucleic acid molecule capable of forming a stable hybrid with the complementary sequence of a nucleic acid molecule encoding the corresponding natural protein. As such, the size of the nucleic acid molecule encoding such a protein homolog is dependent on nucleic acid composition and percent homology between the nucleic acid molecule and complementary sequence as well as upon hybridization conditions per se (e.g., temperature, salt concentration, and formamide concentration). The minimal size of such nucleic acid molecules is typically at least about 12 to about 15 nucleotides in length if the nucleic acid molecules are GC-rich and at least about 15 to about 17 bases in length if they are AT-rich. As such, the minimal size of a nucleic acid molecule used to encode a protease protein homolog of the present invention is from about 12 to about 18 nucleotides in length. There is no limit on the maximal size of such a nucleic acid molecule in that the nucleic acid molecule can include a portion of a gene, an entire gene, or multiple genes, or portions thereof. Similarly, the minimal size of a polymerase protein homolog of the present invention is from about 4 to about 6 amino acids in length, with preferred sizes depending on whether a full-length, multivalent (i.e., fusion protein having more than one domain each of which has a function), or functional portions of such proteins are desired. Polymerase protein homologs of the present invention preferably have activity corresponding to the natural subunit.

The size of the oligonucleotide must also be sufficient for the use of the oligonucleotide in accordance with the present invention. Oligonucleotides identified by the present invention can be used in a variety of applications including, but not limited to, as probes to identify additional polynucleotides, as primers to amplify or extend polynucleotides, as targets for expression analysis, as candidates for targeted mutagenesis and/or recovery, or in agricultural applications to alter polypeptide production or activity. Such agricultural applications include the use of such oligonucleotides in, for example, antisense-, triplex formation-, ribozyme- and/or RNA drug-based technologies. The present invention, therefore, includes such oligonucleotides and methods to enhance economic productivity in a plant by use of one or more of such technologies.

A. Recombinant Molecules

A recombinant vector, which includes at least one polynucleotide identified by the present invention, inserted into any vector capable of delivering the polynucleotide into a host cell, is also contemplated. Such a vector contains heterologous nucleic acid sequences, that is nucleic acid sequences that are not naturally found adjacent to polynucleotides identified by the present invention and that preferably are derived from a species other than the species from which the polynucleotide(s) are derived. As used herein, a derived polynucleotide is one that is identical or similar in sequence to a polynucleotide or portion of a polynucleotide, but can contain modifications, such as modified bases, backbone modifications, nucleotide changes, and the like. The vector can be either RNA or DNA, either prokaryotic or eukaryotic, and typically is a virus or a plasmid. Recombinant vectors can be used in the cloning, sequencing, and/or otherwise manipulating of polynucleotides identified by the present invention. One type of recombinant vector, referred to herein as a recombinant molecule and described in more detail below, can be used in the expression of polynucleotides identified by the present invention. Preferred recombinant vectors are capable of replicating in the transformed cell.

Isolated polypeptides identified by the present invention can be produced in a variety of ways, including production and recovery of natural polypeptides, production and recovery of recombinant polypeptides, and chemical synthesis of the polypeptides. In one embodiment, an isolated polypeptide identified by the present invention is produced by culturing a cell capable of expressing the polypeptide under conditions effective to produce the polypeptide, and recovering the polypeptide. A preferred cell to culture is a recombinant cell that is capable of expressing the polypeptide, the recombinant cell being produced by transforming a host cell with one or more polynucleotides of the present invention. Transformation of a polynucleotide into a cell can be accomplished by any method by which a polynucleotide can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotides identified by the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells can be either untransformed cells or cells that are already transformed with at least one polynucleotide. Host cells of the present invention either can be endogenously (i.e., naturally) capable of producing polypeptides identified by the present invention or can be capable of producing such polypeptides after being transformed with at least one polynucleotide of the present invention. Host cells can be any cell capable of producing at least one polypeptide identified by the present invention, and include bacterial, fungal (including yeast and rice blast, Magnaporthe grisea), parasite (including nematodes, especially of the genera Xiphinema, Helicotylenchus, and Tylenchlohynchus), insect, other animal and plant cells.

Suitable host viruses to transform include any virus that can be transformed with a polynucleotide of the present invention, including, but not limited to, rice stripe virus, and echinochloa hoja blanca virus.

Non-pathogenic symbiotic bacteria, which are able to live and replicate within plant tissues, so-called endophytes, or non-pathogenic symbiotic bacteria, which are capable of colonizing the phyllosphere or the rhizosphere, so-called epiphytes, are also used. Such bacteria include bacteria of the genera Agrobacterium, Alcaligenes, Azospirillum, Azotobacter, Bacillus, Clavibacter, Enterobacter, Erwinia, Flavobacter, Klebsiella, Pseudomonas, Rhizobium, Serratia, Streptomyces and Xanthomonas. Symbiotic fungi, such as Trichoderma and Gliocladium are also possible hosts for expression of the inventive nucleotide sequences for the same purpose.

A recombinant cell is preferably produced by transforming a host cell with one or more recombinant molecules, each comprising one or more polynucleotides identified by the present invention operatively linked to an expression vector containing one or more transcription control sequences. The phrase “operatively linked” refers to insertion of a polynucleotide into an expression vector in a manner such that the molecule is able to be expressed in the correct reading frame when transformed into a host cell. As used herein, an expression vector is a DNA or RNA vector that is capable of transforming a host cell and of effecting expression of a specified polynucleotide. Preferably, the expression vector is also capable of replicating within the host cell. Expression vectors can be either prokaryotic or eukaryotic, and are typically viruses or plasmids. Expression vectors include any vectors that function (i.e., direct gene expression) in recombinant cells of the present invention, including in bacterial, fungal, parasite, insect, other animal, and plant cells. Preferred expression vectors can direct gene expression in bacterial, yeast, fungal, insect and mammalian cells and more preferably in the cell types heretofore disclosed.

Recombinant molecules of the present invention may also (a) contain secretory signals (i.e., signal segment nucleic acid sequences) to enable an expressed polypeptide identified by the present invention to be secreted from the cell that produces the polypeptide and/or (b) contain fusion sequences which lead to the expression of polynucleotides of the present invention as fusion polypeptides. Examples of suitable signal segments and fusion segments encoded by fusion segment nucleic acids are disclosed herein. Eukaryotic recombinant molecules may include intervening and/or untranslated sequences surrounding and/or within the nucleic acid sequences of polynucleotides of the present invention. Suitable signal segments include natural signal segments or any heterologous signal segment capable of directing the secretion of a polypeptide of the present invention. Preferred signal and fusion sequences employed to enhance organ and organelle specific expression include, but are not limited to, arcelin-5, see Goossens, A. et. al. The arcelin-5 Gene of Phaseolus vulgaris directs high seed-specific expression in transgenic Phaseolus acutifolius and Arabidopsis plants. Plant Physiology (1999) 120:1095-1104, phaseolin, see Sengupta-Gopalan, C. et. al. Developmentally regulated expression of the bean beta-phaseolin gene in tobacco seeds. PNAS (1985) 82:3320-3324, hydroxyproline-rich glycoprotein, serpin, see Yan, X. et. al. Gene fusions of signal sequences with a modified beta-glucuronidase gene results in retention of the beta-glucuronidase protein in the secretory pathway/plasma membrane. Plant Physiology (1997) 115:915-924, N-acetyl glucosaminyl transferase 1, see Essl, D. et. al. The N-terminal 77 amino acids from tobacco N-acetylglucosaminyltransferase I are sufficient to retain reporter protein in the Golgi apparatus of Nicotiana benthamiana cells. Febs Letters (1999) 453 (1-2):169-73, albumin, see Vandekerckhove, J. et. al. Enkephalins produced in transgenic plants using modified 2S seed storage proteins. BioTechnology 7:929-932 (1989) and PR1, see Pen, J. et. al. Efficient production of active industrial enzymes in plants. Industrial Crops and Prod. (1993) 1:241-250.

Coding polynucleotides identified by the present invention can be operatively linked to expression vectors containing regulatory sequences such as transcription control sequences, translation control sequences, origins of replication, and other regulatory sequences that are compatible with the recombinant cell and that control the expression of polynucleotides of the present invention. In particular, recombinant molecules of the present invention include transcription control sequences. Transcription control sequences are sequences which control the initiation, elongation, and termination of transcription. Included are those transcription control sequences which are sufficient to render promoter-dependent gene expression controllable for cell-type specific, tissue-specific or inducible by external signals or agents; such elements may be located in the 5′ or 3′ regions of the native gene. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, fungal, insect and mammalian cells, such as, but not limited to, tac, lac, trp, trc, oxy-pro, omp/lpp, rrnB, bacteriophage lambda (λ) (such as λp_{L} and λp_{R} and fusions that include such promoters), bacteriophage T7, T7lac, bacteriophage T3, bacteriophage SP6, bacteriophage SP01, metallothionein, α-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, poxvirus, adenovirus, cytomegalovirus (such as intermediate early promoters, simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

Particularly preferred transcription control sequences are plant transcription control sequences. The choice of transcription control sequence will vary depending on the temporal and spatial requirements for expression, and also depending on the target species. Thus, expression of the nucleotide sequences identified by this invention in any plant organ (leaves, roots, seedlings, immature or mature reproductive structures, etc.) or at any stage of plant development is preferred. Although many transcription control sequences from dicotyledons have been shown to be operational in monocotyledons and vice versa, ideally dicotyledonous transcription control sequences are selected for expression in dicotyledons, and monocotyledonous promoters for expression in monocotyledons. However, there is no restriction to the provenance of selected transcription control sequences; it is sufficient that they are operational in driving the expression of the nucleotide sequences in the desired cell.

Preferred transcription control sequences that are expressed constitutively include but are not limited to promoters from genes encoding actin or ubiquitin and the CaMV 35S and 19S promoters. The nucleotide sequences identified by this invention can also be expressed under the regulation of promoters that are chemically regulated. This enables the corresponding polypeptide to be synthesized only when the crop plants are treated with the inducing chemicals. Preferred technology for chemical induction of gene expression is detailed in the published application EP 0 332 104 (to Ciba-Geigy) and U.S. Pat. No. 5,614,395. A preferred promoter for chemical induction is the tobacco PR-1a promoter.

A preferred category of promoters is that which is induced by the physiological state of the plant (i.e. wound inducible, water-stress inducible, salt-stress inducible, disease inducible, and the like). Numerous promoters have been described which are expressed at wound sites and also at the sites of phytopathogen infection. Ideally, such a promoter should only be active locally at the sites of infection, and in this way the polypeptides only accumulate in cells in which the accumulation is desired. Preferred promoters of this kind include those described by Stanford et al. Mol. Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Preferred tissue-specific expression patterns include but are not limited to green tissue specific, root specific, stem specific, and flower specific. Promoters suitable for expression in green tissue include many which regulate genes involved in photosynthesis and many of these have been cloned from both monocotyledons and dicotyledons. A preferred promoter is the maize PEPC promoter from the phosphoenol carboxylase gene (Hudspeth & Grula, Plant Molec. Biol. 12: 579-589 (1989)). A preferred promoter for root specific expression is that described by de Framond (FEBS 290: 103-106 (1991); EP 0 452 269 to Ciba-Geigy). A preferred stem specific promoter is that described in U.S. Pat. No. 5,625,136 (to Ciba-Geigy) and which drives expression of the maize trpA gene.

A recombinant molecule of the present invention is a molecule that can include at least one of any polynucleotide heretofore described operatively linked to at least one of any transcription control sequence capable of effectively regulating expression of the polynucleotide(s) in the cell to be transformed, examples of which are disclosed herein.

A recombinant cell of the present invention includes any cell transformed with at least one of any polynucleotide identified by the present invention. Suitable and preferred polynucleotides as well as suitable and preferred recombinant molecules with which to transfer cells are disclosed herein.

Recombinant cells of the present invention can also be co-transformed with one or more recombinant molecules including polynucleotides encoding one or more polypeptides identified by the present invention and one or more other polypeptides useful when expressed in plants.

It may be appreciated by one skilled in the art that use of recombinant DNA technologies can improve expression of transformed polynucleotides by manipulating, for example, the number of copies of the polynucleotides within a host cell, the efficiency with which those polynucleotides are transcribed, the efficiency with which the resultant transcripts are translated, and the efficiency of post-translational modifications. Recombinant techniques useful for increasing the expression of polynucleotides identified by the present invention include, but are not limited to, operatively linking polynucleotides to high-copy number plasmids, integration of the polynucleotides into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotides of the present invention to correspond to the codon usage of the host cell, deletion of sequences that destabilize transcripts, and use of control signals that temporally separate recombinant cell growth from recombinant enzyme production during fermentation. The activity of an expressed recombinant polypeptide identified by the present invention may be improved by fragmenting, modifying, or derivatizing polynucleotides encoding such a polypeptide.

Recombinant cells of the present invention can be used to produce one or more polypeptides of the present invention by culturing such cells under conditions effective to produce such a polypeptide, and recovering the polypeptide. Effective conditions to produce a polypeptide include, but are not limited to, appropriate media, bioreactor, temperature, pH and oxygen conditions that permit polypeptide production. An appropriate, or effective, medium refers to any medium in which a cell of the present invention, when cultured, is capable of producing a polypeptide identified by the present invention. Such a medium is typically an aqueous medium comprising assimilable carbon, nitrogen and phosphate sources, as well as appropriate salts, minerals, metals and other nutrients, such as vitamins. The medium may comprise complex nutrients or may be a defined minimal medium. Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant polypeptides of the present invention may either remain within the recombinant cell; be secreted into the fermentation medium; be secreted into a space between two cellular membranes, such as the periplasmic space in E. coli; or be retained on the outer surface of a cell or viral membrane.

The phrase “recovering the polypeptide” refers simply to collecting the whole fermentation medium containing the polypeptide and need not imply additional steps of separation or purification. Polypeptides identified by the present invention can be purified using a variety of standard polypeptide purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Polypeptides identified by the present invention are preferably retrieved in “substantially pure” form. As used herein, “substantially pure” refers to a purity that allows for the effective use of the polypeptide as a diagnostic or test compound, and means, with increasing preference, at least 50%, 60%, 70%, 80%, 90%, 95%, or 98% homogeneous.

With regard to plant polynucleotides identified by the present invention, particularly preferred recombinant cells are plant cells. By “plant cell” is meant any self-propagating cell bounded by a semi-permeable membrane and containing a plastid. Such a cell also requires a cell wall if further propagation is desired. Plant cell, as used herein includes, without limitation, algae, cyanobacteria, seeds, suspension cultures, embryos, meristematic regions, callus tissue, leaves, roots, shoots, gametophytes, sporophytes, pollen, and microspores.

In a particularly preferred embodiment, at least one of the polypeptides or an allele thereof, of the invention is expressed in a higher organism, e.g., a plant. In this case, transgenic plants express effective amounts of the polypeptides to exhibit a unique, enhanced, or altered trait with commercial value. A nucleotide sequence identified by the present invention is inserted into an expression cassette, which is then preferably stably integrated in the genome of said plant. In another preferred embodiment, the nucleotide sequence is included in a non-pathogenic self-replicating virus. Plants transformed in accordance with the present invention may be monocots or dicots and include, but are not limited to, maize, wheat, barley, rye, millet, chickpea, lentil, flax, olive, fig almond, pistachio, walnut, beet, parsnip, citrus fruits, including, but not limited to, orange, lemon, lime, grapefruit, tangerine, minneola, and tangelo, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, and woody plants such as coniferous and deciduous trees.

Once a desired nucleotide sequence has been transformed into a particular plant species, it may be propagated in that species or moved into other varieties of the same species, particularly including commercial varieties, using traditional breeding techniques.

Accordingly, the present invention provides a method for producing a transfected plant cell or transgenic plant comprising the steps of a) transfecting a plant cell to contain a heterologous DNA segment encoding a protein and derived from a polynucleotide identified by the present invention and not native to said cell (the polynucleotide indeed could be native but the expression pattern could be developmentally altered, still leading to the preferred effect); wherein said polynucleotide is operably linked to a promoter that can be used effectively for expression of transgenic proteins; b) optionally growing and maintaining said cell under conditions whereby a transgenic plant is regenerated therefrom; c) optionally growing said transgenic plant under conditions whereby said DNA is expressed, whereby the total amount of identified polypeptide in said plant is altered. In a preferred embodiment, the method further comprises the step of obtaining and growing additional generations of descendants of said transgenic plant which comprise said heterologous DNA segment wherein said heterologous DNA segment is expressed. As used herein, “heterologous DNA”, or in some cases, “transgene” refers to foreign genes or polynucleotides, or additional, or modified versions of native or endogenous genes or polynucleotides (perhaps driven by different promoters) in order to alter the traits of a plant in a specific manner.

The invention also provides plant cells which comprise heterologous DNA encoding a polypeptide identified by the present invention. In a preferred embodiment, the transgenic plant cell is a propagation material of a transgenic plant. The present invention also provides a transfected host cell comprising a host cell transfected with a construct comprising a promoter, enhancer or intron polynucleotide from an evolutionarily significant polynucleotide, and a polynucleotide encoding a reporter protein.

The present invention also provides a method of providing a unique, enhanced, or altered trait in a plant comprising: producing a transfected plant cell having a transgene encoding a polypeptide identified by the present invention. In some embodiments, the expression of the transgene produces an RNA that may interfere with a native gene such that the expression of the native gene is either eliminated or reduced, resulting in a useful outcome.

The invention also provides a transgenic plant containing heterologous DNA which encodes a polypeptide identified by the present invention that is expressed in plant tissue, including expression in a vector introduced into the plant.

The present invention also provides an isolated polynucleotide which includes a transcription control element operably linked to a polynucleotide that encodes a gene identified by the present invention in plant tissue. In a preferred embodiment, the transcription control element is the promoter native to the identified gene.

The present invention also provides a method of making a transfected cell comprising a) identifying a polynucleotide according the the method of the present invention in a domesticated plant; b) using said polynucleotide to identify a non-polypeptide coding sequence that may be a transcription or translation regulatory element, enhancer, intron or other 5′ or 3′ flanking sequence; c) assembling a construct comprising said non-polypeptide coding sequence and a polynucleotide encoding a reporter protein; and d) transfecting said construct into a host cell. The present invention also provides a transfected cell produced according to this method. In one embodiment, the host cell is a plant cell, and the method further comprises the step of growing and maintaining the cell under conditions suitable for regenerating a transgenic plant. Also provided is a transgenic plant produced by the method.

A nucleotide sequence identified by this invention is preferably expressed in transgenic plants, thus causing the biosynthesis of the corresponding polypeptide in the transgenic plants. In this way, transgenic plants with characteristics related to improved economic productivity are generated. For their expression in transgenic plants, the nucleotide sequences of the invention may require modification and optimization. Although preferred gene sequences may be adequately expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17. 477498 (1989)). All changes required to be made within the nucleotide sequences such as those described above are made using well known techniques of site directed mutagenesis, PCR, and synthetic gene construction using the methods described in the published patent applications EP 0 385 962 (to Monsanto), EP 0 359 472 (to Lubrizol), and WO 93/07278 (to Ciba-Geigy).

For efficient initiation of translation, sequences adjacent to the initiating methionine may require modification. For example, they can be modified by the inclusion of sequences known to be effective in plants. Joshi has suggested an appropriate consensus for plants (NAR 15: 6643-6653 (1987)) and Clontech suggests a further consensus translation initiator (1993/1994 catalog, page 210). These consensuses are suitable for use with the nucleotide sequences of this invention. The sequences are incorporated into constructions comprising the nucleotide sequences, up to and including the ATG (while leaving the second amino acid unmodified), or alternatively up to and including the GTC subsequent to the ATG (with the possibility of modifying the second amino acid of the transgene).

Expression of the nucleotide sequences in transgenic plants is driven by transcription control elements shown to be functional in plants. Transformation of plants with a polynucleotide under the control of these regulatory elements provides for controlled expression in the transformed plant. Such transcription control elements have been described above. In addition to the selection of a suitable initiator of transcription, constructions for expression of polypeptides in plants require an appropriate transcription terminator to be attached downstream of the heterologous nucleotide sequence. Several such terminators are available and known in the art (e.g. tm1 from CaMV, E9 from rbcS). Any available terminator known to function in plants can be used in the context of this invention.

Numerous other sequences can be incorporated into expression cassettes described in this invention. These include sequences which have been shown to enhance expression such as intron sequences (e.g. from Adh1 and bronze1) and viral leader sequences (e.g. from TMV, MCMV and AMV).

The present invention also provides a method of providing controllable yield in a transgenic plant comprising: a) producing a transfected plant cell having a transgene containing the identified gene under the control of a promoter providing controllable expression of the identified gene; and b) growing a transgenic plant from the transgenic plant cell wherein the identified transgene is controllably expressed in the transgenic plant. In one embodiment, the identified gene is expressed using a tissue-specific or cell type-specific promoter, or by a promoter that is activated by the introduction of an external signal or agent, such as a chemical signal or agent.

It may be preferable to target expression of the nucleotide sequences of the present invention to different cellular localizations in the plant. In some cases, localization in the cytosol may be desirable, whereas in other cases, localization in some subcellular organelle may be preferred. Subcellular localization of heterologous DNA encoded polypeptides is undertaken using techniques well known in the art. Typically, the DNA encoding the target peptide from a known organelle-targeted gene product is manipulated and fused upstream of the nucleotide sequence. Many such target sequences are known for the chloroplast and their functioning in heterologous constructions has been shown. The expression of the nucleotide sequences of the present invention is also targeted to the endoplasmic reticulum or to the vacuoles of the host cells. Techniques to achieve this are well-known in the art.

Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construction of interest may be preferred. In the case of direct gene transfer, transformation with a single DNA species or co-transformation can be used (Schocher et al. Biotechnology 4: 1093-1096 (1986)). For both direct gene transfer and Agrobacterium-mediated transfer, transformation is usually (but not necessarily) undertaken with a selectable marker which may provide resistance to an antibiotic (kanamycin, hygromycin or methotrexate) or a herbicide (basta). The choice of selectable marker is not, however, critical to the invention.

In another preferred embodiment, a nucleotide sequence of the present invention is directly transformed into the plastid genome. A major advantage of plastid transformation is that plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Natl. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-polypeptide antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19: 4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant polypeptide. In a preferred embodiment, a nucleotide sequence of the present invention is inserted into a plastid targeting vector and transformed into the plastid genome of a desired plant host. Plants homoplastic for plastid genomes containing a nucleotide sequence of the present invention are obtained, and are preferentially capable of high expression of the nucleotide sequence.

The present invention also provides a method of identifying a plant yield-related gene comprising: a) providing a plant tissue sample; b) introducing into the plant tissue sample a candidate plant yield-related gene; c) expressing the candidate plant yield-related gene within the plant tissue sample; and d) determining whether the plant tissue sample exhibits change in yield response, whereby a change in response identifies a plant yield-related gene. The present invention also provides plant yield-related genes isolated according to the method.

Yield response, as used herein, is measured by techniques well known to those skilled in the art. In the cereals, yield response is determined, for example, by one or more of the following metrics, grain weight, grain length, grain weight/1000 grain, size of panicle, number of panicles, and number of grains/panicle.

In another embodiment, this method can be used for mammalian genes, to detect medically important genes such as those involved in disease resistance or susceptibility. For example, after the gene defect causing sickle cell disease was identified, researchers demonstrated evolutionary bottlenecking in populations that had been subject to sickle-cell disease. In a case where a defined human population exhibits resistance or susceptibility to a particular disease, the methods herein could quickly reveal which genes confer resistance or susceptibility. Knowledge of these genes could then lead to therapeutics.

B. Screening Methods

The present invention also provides screening methods using the polynucleotides and polypeptides identified and characterized using the above-described methods. These screening methods are useful for identifying agents which may modulate the function(s) of the polynucleotides or polypeptides in a manner that would be useful for enhancing or diminishing a characteristic in a domesticated organism. Generally, the methods entail contacting at least one agent to be tested with either a transgenic organism or cell that has been transfected with a polynucleotide sequence identified by the methods described above, or a preparation of the polypeptide encoded by such polynucleotide sequence, wherein an agent is identified by its ability to modulate function of either the polynucleotide sequence or the polypeptide. For example, an agent can be a compound that is applied or contacted with a domesticated plant or animal to induce expression of the identified gene at a desired time. Specifically in regard to plants, an agent could be used to induce flowering at an appropriate time.

As used herein, the term “agent” means a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

To “modulate function” of a polynucleotide or a polypeptide means that the function of the polynucleotide or polypeptide is altered when compared to not adding an agent. Modulation may occur on any level that affects function. A polynucleotide or polypeptide function may be direct or indirect, and measured directly or indirectly. A “function” of a polynucleotide includes, but is not limited to, replication, translation, and expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function. The ways that an effective agent can act to modulate the expression of a polynucleotide include, but are not limited to 1) modifying binding of a transcription factor to a transcription factor responsive element in the polynucleotide; 2) modifying the interaction between two transcription factors necessary for expression of the polynucleotide; 3) altering the ability of a transcription factor necessary for expression of the polynucleotide to enter the nucleus; 4) inhibiting the activation of a transcription factor involved in transcription of the polynucleotide; 5) modifying a cell-surface receptor which normally interacts with a ligand and whose binding of the ligand results in expression of the polynucleotide; 6) inhibiting the inactivation of a component of the signal transduction cascade that leads to expression of the polynucleotide; and 7) enhancing the activation of a transcription factor involved in transcription of the polynucleotide.

A “function” of a polypeptide includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

Generally, the choice of agents to be screened is governed by several parameters, such as the particular polynucleotide or polypeptide target, its perceived function, its three-dimensional structure (if known or surmised), and other aspects of rational drug design. Techniques of combinatorial chemistry can also be used to generate numerous permutations of candidates. Those of skill in the art can devise and/or obtain suitable agents for testing.

The in vivo screening assays described herein may have several advantages over conventional drug screening assays: 1) if an agent must enter a cell to achieve a desired therapeutic effect, an in vivo assay can give an indication as to whether the agent can enter a cell; 2) an in vivo screening assay can identify agents that, in the state in which they are added to the assay system are ineffective to elicit at least one characteristic which is associated with modulation of polynucleotide or polypeptide function, but that are modified by cellular components once inside a cell in such a way that they become effective agents; 3) most importantly, an in vivo assay system allows identification of agents affecting any component of a pathway that ultimately results in characteristics that are associated with polynucleotide or polypeptide function.

In general, screening can be performed by adding an agent to a sample of appropriate cells which have been transfected with a polynucleotide identified using the methods of the present invention, and monitoring the effect, i.e., modulation of a function of the polynucleotide or the polypeptide encoded within the polynucleotide. The experiment preferably includes a control sample which does not receive the candidate agent. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, the interactions of the cells when exposed to infectious agents, and the ability of the cells to interact with other cells or compounds. Differences between treated and untreated cells indicate effects attributable to the candidate agent. Optimally, the agent has a greater effect on experimental cells than on control cells.

Appropriate host cells include, but are not limited to, eukaryotic cells, preferably mammalian cells. The choice of cell will at least partially depend on the nature of the assay contemplated.

To test for agents that upregulate the expression of a polynucleotide, a suitable host cell transfected with a polynucleotide of interest, such that the polynucleotide is expressed (as used herein, expression includes transcription and/or translation) is contacted with an agent to be tested. An agent would be tested for its ability to result in increased expression of mRNA and/or polypeptide. Methods of making vectors and transfection are well known in the art. “Transfection” encompasses any method of introducing the exogenous sequence, including, for example, lipofection, transduction, infection or electroporation. The exogenous polynucleotide may be maintained as a non-integrated vector (such as a plasmid) or may be integrated into the host genome.

To identify agents that specifically activate transcription, transcription regulatory regions could be linked to a reporter gene and the construct added to an appropriate host cell. As used herein, the term “reporter gene” means a gene that encodes a gene product that can be identified (i.e., a reporter protein). Reporter genes include, but are not limited to, alkaline phosphatase, chloramphenicol acetyltransferase, β-galactosidase, luciferase and green fluorescence protein (GFP). Identification methods for the products of reporter genes include, but are not limited to, enzymatic assays and fluorimetric assays. Reporter genes and assays to detect their products are well known in the art and are described, for example in Ausubel et al. (1987) and periodic updates. Reporter genes, reporter gene assays, and reagent kits are also readily available from commercial sources. Examples of appropriate cells include, but are not limited to, fungal, yeast, mammalian, and other eukaryotic cells. A practitioner of ordinary skill will be well acquainted with techniques for transfecting eukaryotic cells, including the preparation of a suitable vector, such as a viral vector; conveying the vector into the cell, such as by electroporation; and selecting cells that have been transformed, such as by using a reporter or drug sensitivity element. The effect of an agent on transcription from the regulatory region in these constructs would be assessed through the activity of the reporter gene product.

Besides the increase in expression under conditions in which it is normally repressed mentioned above, expression could be decreased when it would normally be expressed. An agent could accomplish this through a decrease in transcription rate and the reporter gene system described above would be a means to assay for this. The host cells to assess such agents would need to be permissive for expression.

Cells transcribing mRNA (from the polynucleotide of interest) could be used to identify agents that specifically modulate the half-life of mRNA and/or the translation of mRNA. Such cells would also be used to assess the effect of an agent on the processing and/or post-translational modification of the polypeptide. An agent could modulate the amount of polypeptide in a cell by modifying the turn-over (i.e., increase or decrease the half-life) of the polypeptide. The specificity of the agent with regard to the mRNA and polypeptide would be determined by examining the products in the absence of the agent and by examining the products of unrelated mRNAs and polypeptides. Methods to examine mRNA half-life, protein processing, and protein turn-over are well know to those skilled in the art.

In vivo screening methods could also be useful in the identification of agents that modulate polypeptide function through the interaction with the polypeptide directly. Such agents could block normal polypeptide-ligand interactions, if any, or could enhance or stabilize such interactions. Such agents could also alter a conformation of the polypeptide. The effect of the agent could be determined using immunoprecipitation reactions. Appropriate antibodies would be used to precipitate the polypeptide and any protein tightly associated with it. By comparing the polypeptides immunoprecipitated from treated cells and from untreated cells, an agent could be identified that would augment or inhibit polypeptide-ligand interactions, if any. Polypeptide-ligand interactions could also be assessed using cross-linking reagents that convert a close, but noncovalent interaction between polypeptides into a covalent interaction. Techniques to examine protein—protein interactions are well known to those skilled in the art. Techniques to assess protein conformation are also well known to those skilled in the art.

It is also understood that screening methods can involve in vitro methods, such as cell-free transcription or translation systems. In those systems, transcription or translation is allowed to occur, and an agent is tested for its ability to modulate function. For an assay that determines whether an agent modulates the translation of mRNA or a polynucleotide, an in vitro transcription/translation system may be used. These systems are available commercially and provide an in vitro means to produce mRNA corresponding to a polynucleotide sequence of interest After mRNA is made, it can be translated in vitro and the translation products compared. Comparison of translation products between an in vitro expression system that does not contain any agent (negative control) with an in vitro expression system that does contain an agent indicates whether the agent is affecting translation. Comparison of translation products between control and test polynucleotides indicates whether the agent, if acting on this level, is selectively affecting translation (as opposed to affecting translation in a general, non-selective or non-specific fashion). The modulation of polypeptide function can be accomplished in many ways including, but not limited to, the in vivo and in vitro assays listed above as well as in in vitro assays using protein preparations. Polypeptides can be extracted and/or purified from natural or recombinant sources to create protein preparations. An agent can be added to a sample of a protein preparation and the effect monitored; that is whether and how the agent acts on a polypeptide and affects its conformation, folding (or other physical characteristics), binding to other moieties (such as ligands), activity (or other functional characteristics), and/or other aspects of protein structure or functions is considered to have modulated polypeptide function.

In an example for an assay for an agent that binds to a polypeptide encoded by a polynucleotide identified by the methods described herein, a polypeptide is first recombinantly expressed in a prokaryotic or eukaryotic expression system as a native or as a fusion protein in which a polypeptide (encoded by a polynucleotide identified as described above) is conjugated with a well-characterized epitope or protein. Recombinant polypeptide is then purified by, for instance, immunoprecipitation using appropriate antibodies or anti-epitope antibodies or by binding to immobilized ligand of the conjugate. An affinity column made of polypeptide or fusion protein is then used to screen a mixture of compounds which have been appropriately labeled. Suitable labels include, but are not limited to fluorochromes, radioisotopes, enzymes and chemiluminescent compounds. The unbound and bound compounds can be separated by washes using various conditions (e.g. high salt, detergent) that are routinely employed by those skilled in the art. Non-specific binding to the affinity column can be minimized by pre-clearing the compound mixture using an affinity column containing merely the conjugate or the epitope. Similar methods can be used for screening for an agent(s) that competes for binding to polypeptides. In addition to affinity chromatography, there are other techniques such as measuring the change of melting temperature or the fluorescence anisotropy of a protein which will change upon binding another molecule. For example, a BIAcore assay using a sensor chip (supplied by Pharmacia Biosensor, Stitt et al. (1995) Cell 80: 661-670) that is covalently coupled to polypeptide may be performed to determine the binding activity of different agents.

It is also understood that the in vitro screening methods of this invention include structural, or rational, drug design, in which the amino acid sequence, three-dimensional atomic structure or other property (or properties) of a polypeptide provides a basis for designing an agent which is expected to bind to a polypeptide. Generally, the design and/or choice of agents in this context is governed by several parameters, such as side-by-side comparison of the structures of a domesticated organism's and homologous ancestral polypeptides, the perceived function of the polypeptide target, its three-dimensional structure (if known or surmised), and other aspects of rational drug design. Techniques of combinatorial chemistry can also be used to generate numerous permutations of candidate agents.

Also contemplated in screening methods of the invention are transgenic animal and plant systems, which are known in the art.

The screening methods described above represent primary screens, designed to detect any agent that may exhibit activity that modulates the function of a polynucleotide or polypeptide. The skilled artisan will recognize that secondary tests will likely be necessary in order to evaluate an agent further. For example, a secondary screen may comprise testing the agent(s) in an infectivity assay using mice and other animal models (such as rat), which are known in the art or the domesticated plant or animal itself. In addition, a cytotoxicity assay would be performed as a further corroboration that an agent which tested positive in a primary screen would be suitable for use in living organisms. Any assay for cytotoxicity would be suitable for this purpose, including, for example the MTT assay (Promega).

The invention also includes agents identified by the screening methods described herein.

### EXAMPLES

### Example 1

### Genomic DNA Sequencing

A QTL that controls a trait of interest in modern domesticated rice (Oryza saliva) is chosen, for example, QTL gw3.1, the QTL that controls more than 50% of the variation in 1000-grain weight (Xiao, et al., Genetics. 1998 150 (2):899-909), which is an important yield trait. Genomic DNA is prepared from fifteen strains of rice, by methods well known to those of ordinary skill in the art. Suitable primers are designed based upon published genomic sequence of modern rice, available from public databases such as GenBank. A person of ordinary skill in the art can design such primers. These primers are used in PCR to amplify some or all of the QTL of interest, from the ten to fifteen strains and/or individuals of a single strain of rice from which genomic DNA was prepared. A person of ordinary skill in the art can perform this amplification. The amplified PCR products are then sequenced by methods well known to those of ordinary skill in the art.

### Example 2

### Evolutionary Bottleneck Analysis

Homologous DNA sequences of gw3.1 from each of fifteen strains and/or individuals of a single strain of rice are aligned, by methods well known to those skilled in the art. Once homologous sequences are aligned, then the number of nucleotide differences/site (π) can be estimated. The formula used for determining it for a number of sequences (n) is

$$π = 1 / [ n ( n - 1 ) / 2 ] {\sum }_{i < j} Πⅈj / L$$

where i and j represent any two sequences being compared in a series of sequences and L=sequence length.

Regions of the QTL with low π estimates are chosen. (These are the candidates for genes of agricultural value.) No conclusive data are available for rice regarding the expected range of rice-specific π values. As π values are determined for more and more rice sequences, the range of π values as well as the unusually low π values of interest will be refined.

As π values are estimated sequentially (or successively) along the DNA sequence, an overlapping strategy is useful, in which, after estimating π values along the sequences, the frame of reference is shifted by a predetermined number of base pairs, say 50 bp. As little data on expected values of π currently exists, the optimal number of base pairs to shift to a new frame of reference must be determined empirically for each species examined. Similarly, the optimal length of sequence to estimate π values will also be determined for each species examined.

Regions determined to have low values of π are candidates for controlling grain weight in rice. These regions will be characterized by methods well known to those of ordinary skill in the art, as being regulatory, protein-coding, and so on.

1. A method for identifying a polynucleotide sequence, wherein the polynucleotide sequence may be associated with a commercially or aesthetically relevant trait, comprising:
a) aligning homologous nucleotide sequences of at least two individual organisms, wherein said at least two individual organisms are selected from the group consisting of individual organisms of a single strain, individual organisms of different strains, individual organisms of the same species, individual organisms of different species, and any combination of the foregoing, wherein one nucleotide sequence is associated with an individual organism exhibiting said commercially or aesthetically relevant trait; and
b) detecting a region of polynucleotide sequence for which the number of nucleotide differences/site indicates an evolutionary bottleneck;
whereby a polynucleotide sequence associated with a commercially or aesthetically relevant trait of said organism may be identified.

2. (canceled)

3. A method for identifying a polynucleotide sequence encoding a polypeptide, wherein the polypeptide may be associated with a commercially or aesthetically relevant trait comprising:
a) aligning homologous protein-coding nucleotide sequences of at least two individual organisms, wherein said at least two individual organisms are selected from the group consisting of individual organisms of a single strain, individual organisms of different strains, individual organisms of the same species, individual organisms of different species, and any combination of the foregoing, wherein one nucleotide sequence encodes a polypeptide associated with an domesticated organism exhibiting said commercially or aesthetically relevant trait; and
b) detecting a region of polynucleotide sequence for which the number of nucleotide differences/site indicates an evolutionary bottleneck;
whereby a polynucleotide sequence associated with a commercially or aesthetically relevant trait of said organism may be identified.

4. (canceled)

5. The method of claim 1, further comprising
c) determining that the region identified in b) is a non-coding region, wherein the polynucleotide sequence is a regulatory element.

6. The method according to claim 1, wherein the identifying the number of nucleotide differences/site is calculated by
$$π = 1 / [ n ( n - 1 ) / 2 ] {\sum }_{i < j} Πⅈj / L ,$$
n is number of sequences, where i and j represent any two sequences being compared in a series of sequences and L=sequence length.

7. The method according to claim 1, further comprising determining if the region displays a signature of positive selection.

8. The method of claim 97, wherein said determining comprises calculating a Ka/Ks value.

9. The method according to claim 1, wherein the method is performed in an automated pipeline.

10. The method according to claim 1, wherein the at least two strains and/or individuals of a single strain is at least ten strains and/or individuals of a single strain.

11. The method of claim 10 wherein the at least two strains and/or individuals of a single strain is at least fifteen strains and/or individuals of a single strain.

12. A method for identifying an agent which may modulate a commercially or aesthetically relevant trait that is unique, enhanced or altered in the domesticated organism as compared to other domesticated or ancestral species of the domesticated organism, said method comprising contacting at least one candidate agent with a cell, model system or transgenic plant or animal that expresses a polynucleotide sequence that is an evolutionary bottleneck, wherein the agent is identified by its ability to modulate function of the polypeptide encoded by the polynucleotide.

13. A method for correlating a nucleotide sequence which is an evolutionary bottleneck to a commercially or aesthetically relevant trait that is unique, enhanced or altered in a domesticated organism, comprising:
a) identifying a nucleotide sequence which is an evolutionary bottleneck; and
b) analyzing the functional effect of the presence or absence of the identified sequence in the domesticated organism or in a model system.

14. The method of claim 1, wherein the polynucleotide sequence is a regulatory element.

15. The method according to claim 3, wherein the identifying the number of nucleotide differences/site is calculated by
$$π = 1 / [ n ( n - 1 ) / 2 ] {\sum }_{i < j} Πⅈj / L ,$$
n is number of sequences, where i and j represent any two sequences being compared in a series of sequences and L=sequence length.

16. The method according to claim 3, further comprising determining if the region displays a signature of positive selection.

17. The method of claim 16, wherein said determining comprises calculating a Ka/Ks value.

18. The method according to claim 3, wherein the method is performed in an automated pipeline.

19. The method according to claim 3, wherein the at least two strains and/or individuals of a single strain is at least ten strains and/or individuals of a single strain.

20. The method of claim 3, wherein the at least two strains and/or individuals of a single strain is at least fifteen strains and/or individuals of a single strain.
